(19) United States
(12) Patent Application Publication
(10) Pub. No.: US 2019/0307659 A1
(43) Pub. Date: Oct. 10, 2019

(54) COSMETIC TENSIONING COMPOSITION

(71) Applicant: Noxell Corporation, Hunt Valley, MD (US)

(72) Inventors: Yong Zhu, Marriottsville, MD (US); Gordon Gerald Guay, Joppa, MD (US)

(21) Appl. No.: 16/322,656

(22) PCT Filed: Sep. 14, 2017
(86) PCT No.: PCT/US2017/051647

§ 371 Date: Feb. 1, 2019

Related U.S. Application Data
(60) Provisional application No. 62/394,418, filed on Sep. 14, 2016.

(51) Int. Cl. A61K 8/25; A61K 8/81; A61Q 19/00; A61Q 19/08; A61Q 1/04; A61Q 1/10; A61Q 5/06; A61K 8/02; A61K 8/04; A61K 8/06

(52) U.S. Cl. CPC A61K 8/25; A61K 2800/43; A61K 8/8182; A61Q 19/001; A61Q 19/08; A61Q 1/04; A61Q 1/10; A61Q 5/06; A61K 8/025; A61K 8/044; A61K 8/06; A61K 8/8135; A61K 8/8152; A61K 2800/412; A61K 2800/594; A61K 8/8158

(57) ABSTRACT

A cosmetic composition having a film-forming tensioning polymer system and at least one hydrophilic non-colloidal particulate agent comprising precipitated silica particles is disclosed. The film forming composition comprises a first non-crosslinking polyamide/polyacrylate copolymer comprising at least one amide monomer; (meth)acrylate monomers; monomers having at least one carboxylic functional group; and monomers having at least one amine functional group. The film forming composition also comprises a second non-crosslinking polyamide copolymer comprising at least one amide; at least one quaternary ammonium containing monomer; and monomers having at least one amine functional group.

### PRIORITY

This application claims the benefit of priority from U.S. Provisional Patent Application No. 62/394,418, filed on Sep. 14, 2016, which is herein incorporated in its entirety by reference.

### FIELD OF THE INVENTION

The present application is directed, generally, to cosmetic compositions having a film-forming tensioning polymer system.

### BACKGROUND OF THE INVENTION

A frequently-stated desire of consumers is a cosmetic product that can temporarily eliminate or reduce the appearance of unwanted skin texture imperfections (e.g., pores, fine lines, and wrinkles) by providing a noticeable skin smoothing effect in an immediate or rapid manner. Attempts have been made to develop new categories of products to improve the appearance of skin without the drawbacks of existing products and procedures. One such family of products can be generally classified as “adhesive, contractile film formers”. Film formers are chemical compositions that when applied to skin, leave a pliable, cohesive and continuous covering. A select group of film formers are also adhesive to skin and even contractile. Wrinkles, in their simplest form, are crevices or valleys in the skin. When an adhesive, contractile film former is applied, the skin at the bottom of the valley or crevice may be pulled to the surface, causing skin look smooth and wrinkle-free. The drawbacks of existing adhesive, contractile film forming products include discomfort caused by the contraction of the skin, irritation of the skin, cracking of the film as the consumer uses her face muscles, incompatibility with other cosmetic products in her regimen, and visibility of the film which is often whitish and noticeable. Curing or reducing one of these problems has, in the past, exacerbated one of the other problems.

Preferably, the skin-tightening benefit would come from a product they already use as part of their daily regimen, such as anti-aging facial or eye area treatments and primers for makeup, or pigmented cosmetics such as tinted moisturizers, foundations, and concealers. However, cosmetics that can effectively produce such a result in a manner that is desirable to consumers are not currently available. In addition, as consumers apply pigmented cosmetics (such as foundations) to enable the visual appearance of natural skin, one of the limitations is that these pigmented systems actually accentuate skin flaws. Also, adding these types of particles to existing skin tensioning systems degrades the effectiveness of the system, since the particles interfere with the film formation.

Therefore, a need still exists for a cosmetic product that can temporarily eliminate or reduce the appearance of unwanted skin texture imperfections.

### SUMMARY

The present invention provides a cosmetic product that gives consumers a means to deliver desired pigment while reducing the appearance of skin flaws. Further, it achieves this skin smoothing effect in a manner that is more natural looking and feeling when applied to a consumer's face or body. These compositions can be useful in a variety of applications, including use as a skin foundation product, a skin care product, a lip product, a hair styling product, and a mascara product. Specifically, there is disclosed a cosmetic composition that provides skin tensioning benefits.

In one embodiment, the present invention provides particulate agents in amounts capable of not only reducing shine of these films, but also minimizes the appearance of skin imperfections, such as uneven skin tone, spots, pores, wrinkles, and fine lines with enhanced skin texture modification and lash curling and lifting under broader relative humidity condition or moisture conditions.

The contractile and skin tensing properties of the film-forming tensioning polymers are typically observed to be largely compromised or degraded by the inclusion of particles either having certain physical or chemical properties (e.g., size, surface energy or charge) incompatible with the polymers, or when these particles are used at too high a concentration in relation to the amount of film-forming polymer in the composition. However, through our experimental studies it has been surprisingly discovered that our contractile performance attributes of the specific polymer systems for a specific type of tensioning polymer system can remain essentially unchanged or even further improved by incorporation of particular types of particles having specific properties. Importantly, this is found to be especially true when the preferred particles are used in a limited proportion with respect to the amount of film-forming tensioning polymers.

The present invention discloses a cosmetic composition having an aqueous phase with a film-forming tensioning polymer system and at least one hydrophilic non-colloidal particulate agent comprising precipitated silica particles, lipophilically treated pigment powders, hydrophobically treated pigment powders, or combinations thereof. The film-forming tensioning polymer system comprises:

a) a first non-crosslinking polyamide/polyacrylate copolymer comprising the following monomer units:
i. at least one amide monomer, including vinyl caprolactam monomers, vinylpyrrolidone monomers, and acrylamide monomers;
ii. (meth)acrylate monomers;
iii. monomers having at least one carboxylic functional group selected from the group consisting of carboxylic esters, carboxylic acids, their salts, or precursors of carboxylate functions, and mixtures thereof; and
iv. monomers having at least one amine functional group including primary, secondary and tertiary amines

b) a second non-crosslinking polyamide copolymer comprising the following monomer units:
i. at least one amide monomer, including vinyl caprolactam monomers, vinylpyrrolidone monomers, and (meth)acrylamide monomers;
ii. at least one quaternary ammonium containing monomer and
iii. monomers having at least one amine functional group including primary, secondary, and tertiary amines.

In one embodiment, the film-forming tensioning polymer system is adhesive.

In another embodiment, the first non-crosslinking polyamide/polyacrylate copolymer comprises a polyvinylcaprolactam/vinylpyrrolidone/dimethylaminoethyl methacrylate copolymer, including it methacrylate hydrolysis products, such as methacrylic acid and corresponding salts.

In yet another embodiment, the second non-crosslinking polyamide copolymer comprises a polyvinylcaprolactam/vinylpyrrolidone/dimethylaminoalkyl methacrymade copolymer; and further comprises a polyvinylcaprolactam/vinylpyrrolidone/dimethylaminopropyl methacrylamide/quaternary methyacrylamidopropyl dimethylalkyl ammonium copolymer.

In one embodiment, the precipitated silica particles have a Median Particle Size from about 1 to about 20μ. In another embodiment, the precipitated silica particles have a Median Particle Size from about 2 to about 15μ. In yet another embodiment, the precipitated silica particles have a Median Particle Size from about 3 to about 5μ. In one embodiment, the precipitated silica particles have a Specific Surface Area (SSA) greater than about 300 M2/g.

In one embodiment, the weight ratio of the precipitated silica particles to the film-forming tensioning polymer system is from about 1:20 to about 1:2. In another embodiment, the weight ratio of the precipitated silica particles to the film-forming tensioning polymer system is from about 1:10 to about 2:5. In yet another embodiment, the weight ratio of the precipitated silica particles to the film-forming tensioning polymer system is from about 3:20 to about 6:20. In one embodiment, the weight ratio of the precipitated silica particles to the film-forming tensioning polymer system is from about 1:5 to about 1:4.

In one embodiment, the precipitated silica particles comprise from about 0.1 to about 10 weight percent of the cosmetic composition. In another embodiment, the precipitated silica particles comprise from about 1 to about 8 weight percent of the cosmetic composition. In yet another embodiment, the precipitated silica particles comprise from about 2 to about 6 weight percent of the cosmetic composition.

In one embodiment, the precipitated silica particles consist of precipitated hydrophilic silica particles. In another embodiment, the precipitated silica particles comprise a mixture of precipitated hydrophilic silica particles and particles selected from the group consisting of hydrophobic particles, additional hydrophilic particles and combinations thereof. In yet another embodiment, the precipitated silica particles comprise hydrophobically treated silicas.

In one embodiment, the precipitated silica particles comprise additional hydrophilic particles selected from the group consisting of precipitated silica, fumed silica, and combinations thereof.

In another embodiment, the cosmetic composition of further comprise hydrophilically treated wax particles. In one embodiment, the hydrophilically treated wax particles are cold process waxes. In another embodiment, the hydrophilic wax particles comprise from about 0.1 to about 12 weight percent of said cosmetic composition. In yet another embodiment, the hydrophilic wax particles comprise from about 1 to about 6 weight percent of said cosmetic composition.

In one embodiment, the cosmetic composition is in the form of a solution, an emulsion or a suspension. In another embodiment, the solution or suspension is aqueous. In yet another embodiment, the emulsion is a water external emulsion or an oil emulsion. In one embodiment, the oil emulsion includes a silicone phase. In another embodiment, the cosmetic composition is included in a water based system.

The cosmetic composition can be used in a variety of applications, including as a skin foundation product, a skin care product, a hair styling product, a lip product, and a mascara product.

### DETAILED DESCRIPTION OF THE INVENTION

All percentages are by weight of the cosmetic composition, unless otherwise specified. All ratios are weight ratios, unless specifically stated otherwise. All numeric ranges are inclusive of narrower ranges; delineated upper and lower range limits are interchangeable to create further ranges not explicitly delineated. The number of significant digits conveys neither limitation on the indicated amounts nor on the accuracy of the measurements. Unless otherwise stated or prescribed, all measurements are understood to be made from about 22-28° C. and at ambient conditions, where “ambient conditions” means conditions under about one atmosphere of pressure and at about 40-50% relative humidity.

### Definitions

The term “apply” or “application” as used in reference to a composition, means to apply or spread the compositions of the present invention onto a substrate such as the human skin surface or epidermis, and human hair or eyelashes.

The term “dermatologically acceptable” as used herein means that the compositions or components described are suitable for use in contact with human skin tissues and in eye areas without undue toxicity, incompatibility, instability, allergic response, and the like.

The term “facial skin surface” as used herein refers to one or more of forehead, periorbital, cheek, perioral, chin, crow' feet, and nose skin surfaces. While facial skin surfaces are of concern and are exemplified herein, other skin surfaces may be treated with the compositions of the present invention, for example, surfaces typically not covered by clothing such as facial skin surfaces, hand and arm skin surfaces, foot and leg skin surfaces, and neck and chest skin surfaces (e.g., décolletage).

“Keratinous tissue,” means keratin-containing tissue layers disposed as the outermost protective covering of mammals which includes, but is not limited to, skin, hair, and nails.

“Mascara” and “mascara composition” mean a liquid, gel, semi-solid, or solid cosmetic composition that is applied to eyelashes to provide an aesthetic benefit or change in appearance such as, the appearance of a color change, a volume change, and/or a length change. Mascara may also be applied to periorbital areas, eyelids and/or eyebrows. The present mascara compositions are formulated for topical application to mammalian keratinous tissue for use in cosmetic products. The methods of using mascara compositions are also included within the meaning of mascara composition.

“Non-colloidal particulate agent” means a particle that does not in general form a stable suspension in a solvent or suspending supporting medium or fluid by itself without suspension aids, such as shears, hydrodynamic interactions, rheology modifiers or thickeners, or other suspending or dispersing solids, due to its large particle size. Typically, non-colloidal particulate agents have particle sizes greater than 1 micron.

“Non-crosslinking polyamide/polyacrylate random copolymer” means any non-crosslinking amide and acrylate monomer containing random copolymer that include the amide and acrylate monomers as side chains. Examples of such amide and acrylate monomer containing copolymers are Styleze 2000, Copolymer 845, Advantage S, Advantage LCA as supplied by ASI, and Ultrahold Strong as supplied by BASF.

“Non-crosslinking polyamide random copolymer” means any non-crosslinking amide monomer containing random copolymer that includes the amide monomers as side chains. Examples of such amide monomer containing copolymers are Styleze W, Aquastyle 300, Styleze CC, Aquaflex SF, as supplied by ASI, Luviquat Hold, and Luviquat Supreme as supplied by BASF.

“Skin foundation” means a skin makeup applied onto the face or body to try and create even, uniform appearance, or to cover flaws, or to change the natural skin tone. Most foundation products are in liquid form, but also include other forms, such as cake, cream, gel, and lotion. Most facial foundation is colored, but also can be transparent or translucent.

“Water-soluble, film-forming polymers” are defined herein to mean polymers which are soluble or dispersible in water, water-cosolvent mixtures (such as ethanol/water), pH adjusted water, and/or tempered solutions of the above to facilitate solubilization or dispersion of the polymers.

### Compositions

The present invention relates to various compositions and, more specifically, to compositions for application to a skin surface. The compositions may be in a wide variety of product forms that include, but are not limited to, solutions, suspensions, lotions, creams, gels, toners, sticks, pencil, sprays, aerosols, ointments, cleansing liquid washes and solid bars, pastes, foams, powders, mousses, wipes, strips, patches, hydrogels, film-forming products, facial and skin masks (with and without insoluble sheet), make-up such as foundations, eye liners, and eye shadows, and the like. The composition form may follow from the particular dermatologically acceptable carrier chosen, if present in the composition.

### Film-Forming Composition

The present invention comprises a film forming composition comprising a first non-crosslinking polyamide/polyacrylate random copolymer comprising at least one amide monomer including vinyl caprolactam monomers, vinylpyrrolidone monomers and acrylamide monomers; (meth)acrylate monomers; monomers having at least one carboxylic functional group selected from the group consisting of carboxylic esters, carboxylic acids and their salts, or precursors of carboxylate functions, and mixtures thereof; and amine functional groups including primary, secondary and tertiary amines. The film forming composition also comprises a second non-crosslinking polyamide random copolymer comprising at least one amide monomer including vinyl caprolactam monomers, vinylpyrrolidone monomers, and (meth)acrylamide monomers, at least one quaternary ammonium containing monomer; and amine functional groups including primary, secondary, and tertiary amines.

Amide monomers that are useful in the present invention include amide monomers with open-chain organic amide functional groups and derivatives. In one embodiment, the preferred amides include acrylamides and methacrylamides.

Commercially available examples of such amide monomers include the monomers in Styleze W. Styleze CC-10, AquaStyle 300 (PQ69), Aquaflex SF40, ViviPrint 141, Conditioneze NT-20 all commercially available from Ashland Specialty Ingredients (ASI); and Ultrahold Strong, Luviset Clear, Luviquat Supreme (PQ68) all available from BASF. Other examples of this type of polymers can be found in Personal Care Product Consult Database (PCPC).

Amide monomers that are useful in the present invention include amide monomers with cyclic amide functional groups and derivatives.

Commercially available examples of such amide monomers include the monomers in Copolymer 845, 937 and 958, Advantage LCA, LCE and S, PVP/VA (W635, 735), Gafquat, Aquaflex SF-40, Styleze W, Aquastyle 300, ViviPrint 141, Conditioneze NT-20, Styleze CC-10 all available from ASI; and Luviquat Supreme, Luviquat UltraCare, Luviquat Hold, Luviquat PQ11, Luviquat HM552, Luviquat Style, Luviquat FC, Luviquat Excellence, Luviset Clear all available from BASF. Other examples of this type of polymers can be found in Personal Care Product Consult Database (PCPC).

Monomers that are useful in the present invention include amine monomers with functional groups and derivatives.

Commercially available examples of such amine monomers include the monomers in Copolymer 845, 937 and 958, Advantage LCA, LCE and S, Gafquat, Aquaflex SF-40, Styleze W, Aquastyle 300, ViviPrint 141, Aquaflex XL-30, Styleze CC-10 all available from ASI; and Luviquat Supreme from BASF. Other examples of this type of polymers can be found in Personal Care Product Consult Database (PCPC).

Monomers that may be useful in the present invention include amine or ether monomers with quaternary ammonium functional groups and derivatives. Commercially available examples of copolymers containing such monomers include Polyquaternium-5, -11, -14, -19, -22, -28, -37, -46, -47, -51, -55, -69, -87 (all available from BASF).

Monomers that may be useful in the present invention include amide or acrylate monomers with quaternary ammonium functional groups and derivatives. Commercially available examples of copolymers containing such monomers include Polyquaternium-4, -5, -7, -8, -9, -11, -12, -13, -18, -28, -33, -36, -37, -45, -47, -49, -52, -53, -55, -63, -64, -68, -69, -85, -89, -91, -109, and others as described PCPC.

Monomers that may be useful in the present invention include monomers with carboxylic acid, salt and ester functional groups and derivatives. In one embodiment, preferred monomers include: acrylates, methacrylates, acrylic acids and their salts, methacrylic acids and their salts. The carboxylate monomers may include precursors of carboxylate functions, such as tert-butyl (meth)acrylates, alkyl-2-amino ethyl esters of (meth)acrylates which give rise to carboxylic functions by hydrolysis (under more stressed pH, temperature conditions, in the presence of catalysts, or other approaches).

Commercially available examples of copolymers containing such monomers include Advantage Plus, LCA, LCE and S, W635 and 735, Copolymer 845, 937 and 958. Aquaflex XL-30. PVP/VA E-735. E-635, E-535 and W-735, Gafquat, Allianz OPT from ASI; Luviquat PQ11, UltraHold Strong, Luviset Shape, Luviflex Soft, Cosmedia SP from BASF. Other examples of these type of polymers can be found in the Personal Care Product Consult Database (PCPC).

The ester/acid/salt/anhydride functional groups may contain one or more types of esters/acids/salts/anhydrides (e.g. esters, acids and/or salts).

In one embodiment, the preferred second copolymer comprises from about 0.1 to about 45 percent of quaternary ammonium containing monomers. In another embodiment, the preferred second copolymer comprises from about 1 to about 10 percent of quaternary ammonium containing monomers.

Polymer structure similarity is a useful criterion for achieving the present invention's unexpected high contraction and improved dry speed synergy. Another criterion is the charge density of the polymers, which can be useful for delivering the unexpected high contraction and fast dry synergy performances.

### Dermatologically Acceptable Carrier

The compositions of the present invention may also comprise a dermatologically acceptable carrier (which may be referred to as “carrier”) for the composition. The phrase “dermatologically acceptable carrier”, as used herein, means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. Non-limiting examples include simple solutions (e.g., aqueous, organic solvent, or oil based), emulsions, suspensions, and solid forms (e.g., gels, sticks, flowable solids, or amorphous materials). In certain embodiments, the dermatologically acceptable carrier is in the form of an emulsion or suspension. Emulsion or suspension may be generally classified as having a continuous aqueous phase (e.g., oil-in-water and water-in-oil-in-water) or a continuous oil phase (e.g., water-in-oil and oil-in-water-in-oil). The oil phase of the present invention may comprise silicone oils, non-silicone oils such as hydrocarbon oils, esters, ethers, and the like, and mixtures thereof.

Emulsions may further comprise an emulsifier. The composition may comprise any suitable percentage of emulsifier to sufficiently emulsify the carrier. Suitable weight ranges include from about 0.1% to about 10% or about 0.2% to about 5% of an emulsifier, based on the weight of the composition. Emulsifiers may be nonionic, anionic or cationic. Suitable emulsifiers are disclosed in, for example, U.S. Pat. Nos. 3,755,560, 4,421,769, and McCutcheon's Detergents and Emulsifiers, North American Edition, pages 317-324 (1986). Suitable emulsions may have a wide range of viscosities, depending on the desired product form.

The carrier may further comprise a thickening agent as are well known in the art to provide compositions having a suitable viscosity and rheological character.

### Pigments and Powders

The compositions of the present invention can comprise from about 5% to about 45%, preferably from about 5% to about 30% of a powder component. In one embodiment, the powder is a pigment powder. The pigments included in the pigment powder component herein may be hydrophobic in nature, or hydrophobically treated. By keeping the level of pigment component low, the entire composition maintains flexibility to accommodate other components which provide spreadability, moisturization, and fresh and light feel. The species and levels of the pigments are selected to provide, for example, shade, coverage, good wear performance, and stability in the composition.

Powders useful for the powder component herein are inorganic and organic powder such as talc, mica, sericite, synthetic fluorphlogopite, pearl pigments such as alumina, barium sulfate, calcium secondary phosphate, calcium carbonate, coverage titanium oxide, finely divided titanium oxide, zirconium oxide, normal particle size zinc oxide, hydroxy apatite, iron oxide, iron titanate, ultramarine blue, Prussian blue, chromium oxide, chromium hydroxide, cobalt oxide, cobalt titanate, titanium oxide coated mica; organic powder such as polyester, polyethylene, polystyrene, methyl methacrylate resin, cellulose, 12-nylon, 6-nylon, styrene-acrylic acid copolymers, polypropylene, vinyl chloride polymer, tetrafluoroethylene polymer, boron nitride, fish scale guanine, laked tar color dyes, and laked natural color dyes. Such pigments may be treated with a hydrophobical treatment agent, including: silicone such as methicone, dimethicone, and perfluoroalkyl silane; fatty material such as stearic acid and disodium hydrogenated glutamate; metal soap such as aluminium dimyristate; aluminium hydrogenated tallow glutamate, hydrogenated lecithin, lauroyl lysine, aluminium salt of perfluoroalkyl phosphate, and aluminium hydroxide as to reduce the activity for titanium dioxide, and mixtures thereof. Such pigments may also be coated with substances considered more hydrophilic such as polysaccharides, caprylyl silane, or polyethylene oxide silane treatments.

Commercially available pigment powder component includes coverage titanium dioxide, such as SI-T-CR-50Z, SI-Titanium Dioxide IS. SA-Titanium Dioxide CR-50, SI-FTL-300 and SA/NAI-TR-10, all of them are available from Miyoshi Kasei iron oxide and cyclopentasiloxane and dimethicone and disodium hydrogenated glutamate: SA/NAI-Y-10/D5(70%)/SA/NAI-R-10/DS(65%)/SA/NAI-B-10/D5(75%) available from Miyoshi Kasei, iron oxide and disodium hydrogenated glutamate: SA/NAI-Y-10/SA/NAI-R-10/SA/NAI-B-10 available from Miyoshi Kasei, iron oxide and methicone: SI Mapico Yellow Light Lemon XLO/SI Pure Red Iron Oxide R-1599/SI Pure Red Iron Oxide R-3098/SI Pure Red Iron Oxide R-4098/SI Black Iron Oxide No. 247 available from Daito Kasei alumina and titanium dioxide and methicone: SI-LTSG30AFLAKE H (5%) LHC available from Miyoshi Kasei, talc and methicone: SI-Talc JA13R LHC available from Miyoshi Kasei, mica and methicone: SI Mica available from Miyoshi Kasei, dimethicone: SA-SB-300 available from Miyoshi Kasei, mica and methicone: SI Sericite available from Miyoshi Kasei, mica and dimethicone: SA Sericite available from Miyoshi Kasci, mica and C9-15 Fluoroalcol Phosphates and Triethoxy Caprylylsilane: FOTS-52 Sericite FSE available from Daito Kasci, Talc and C9-15 Fluoroalcol Phosphates and triethoxy caprylylsilane: FOTS-52 Talc JA-13R available from Daito Kasei, boron nitride and methicone: SI02 Boron Nitride SHP-6 available from Daito Kasei, boron nitride and C9-15 fluoroalcol phosphates and triethoxy caprylylsilane: FOTS-52 Boron Nitride available from Daito Kasci, mica and titanium dioxide and methicone: SI Sericite TI-2 available from Mivoshi Kasei, mica and titanium dioxide and methicone: SI Mica TI-2 available from Miyoshi Kasei, talc and titanium dioxide and methicone: SI Talc TI-2 available from Miyoshi Kasei, lauroyl lysine: AMIHOPE LL available from Ajinomoto, synthetic fluorphlogopite and methicone: PDM-5L(S)/PDM-10L(S)/PDM-20L(S)/PDM-40L(S) available from Topy Industries.

### Non-Colloidal Particulate Agents

The present invention includes non-colloidal particulate agents. In one embodiment, the non-colloidal particulate agents are hydrophilic. In another embodiment, the hydrophilic non-colloidal particulate agent comprises porous precipitated silica particles.

The precipitated porous silica particles of the present invention are useful for enhancing the properties of the current system such as contractile forces and the ability to make a discontinuous film.

The precipitated porous silica particles of the present invention may combine hydrophobically treated non-colloidal particles or pigments. The combinations of hydrophilic and hydrophobic non-colloidal particles or pigments are useful for enhancing the properties of the current system such as humidity tolerance, and flexibility and elasticity of polymer films.

Commercially available non-colloidal hydrophilic particles include precipitated silicas, including those available under the trade name “Spheron” by Presperse, silica shell. MSS by Kobo. Sipernat by Evonik. Hydrophilically modified waxes are also useful, including polyacrylate treated waxes, such as Cold Process Wax by JEEN International under trademark Jeesperse® (CPW-B and CPW-Carnauba). Nylonpowders are also useful.

Commercially available hydrophobically or lipophilically treated non-colloidal particles and pigments include treated pigments and particles available under the trade name Sympholight. Aquasphersabil by Presperse, DSPCS, SPCAT, SPC. Nylon 10-12. CT-2 Nylon SP500, Silica Shell-SH, MSS500-NSS, MSS500N-FS. MST547-FS, SP-10-FS, SILIGHT DS-PDL3. PUlight SDS, BPD and BBO by Kobo. Covalumine by Sensient, and micronized waxes by Micro Powders.

### Contraction

In one embodiment, the film forming composition produces a desired contraction when applied to a Leneta card. When the composition is applied to a Leneta card, the card has a minimum contraction of 10% with minimum synergy of 120% (as described by the “Contraction Test” method below) when the Leneta card is kept at a temperature in the range of 22 to 28° C. and at a relative humidity in the range of about 40% to 50% to measure the contraction.

### Fast Dry

In another embodiment, the film forming composition produces fast dry kinetics when measured using a “Weight Loss Test” method described below. When the composition is applied to a flat hard substrate, such as glass microscope slide, the film has a shorter dry time, as determined by having a Dry Speed (i.e., time required to reach 90% total weight loss) of less than 10 minutes with a Dry Speed synergy of at least about 110%.

### Products

The cosmetic composition of the present invention can be used in a variety of applications, including as a skin foundation product, a skin care product, a mascara product, a hair styling product, a lip product, and a kit. The present invention also encompasses a water based system comprising the cosmetic composition.

The compositions disclosed herein may be used in many end-use applications. Examples include (but are not limited to) a water phase suspension, an oil in water emulsion, a water in oil emulsion, a silicone in water emulsion, a water in silicone emulsion, a Pickering emulsion, and/or an oil phase suspension, or dispersion, and/or kits.

Carrier and/or Oil

The cosmetic composition may include a carrier to help deliver desired components (e.g., the film former, pigments, etc.) to the skin, eyelash or eyelid. In certain embodiments, the cosmetic composition may include a volatile carrier that quickly volatilizes from the surface of the skin, eyelashes or eyelid, leaving the desired components behind. The volatile carrier may be present at 2% to 85%, 10% to 80%, or even 20% to 70% by weight based on the weight of the composition. Nonlimiting examples of suitable volatile carriers include volatile hydrocarbons, volatile alcohols, volatile silicones, and mixtures thereof.

Hydrocarbon oils suitable for use as a carrier in the present cosmetic compositions include those having boiling points in the range of 60-260° C., such as hydrocarbon oils having a carbon chain length of from C8 to C20 (e.g., C8 to C20 isoparaffins). Particularly suitable examples of isoparaffins include those selected from the group consisting of isododecane, isohexadecane, isoeicosane, 2.2,4-trimethylpentane, 2.3-dimethylhexane and mixtures thereof. Isododecane is available from Presperse under the brand name Permethyl 99A. Alcohols suitable for use may include C₁-C₄ monoalcohols, such as ethyl alcohol and isobutyl alcohol.

A volatile silicone fluid may also be used as a carrier herein. Suitable volatile silicone fluids include dimethicone, trimethicone, and cyclomethicones. Nonlimiting examples of commercially available volatile silicones include 244 Fluid, 344 Fluid and 245 Fluid. and/or 345 Fluid from Dow Corning Corporation.

Oils typically used in cosmetics include those selected from the group consisting of polar oils, non-polar oils, volatile oils, non-volatile oils and mixtures thereof. These oils may be saturated or unsaturated, straight or branched, aliphatic or aromatic hydrocarbons. Preferred oils include non-polar volatile hydrocarbons including isodecane (such as Permethyl-99A®, available from Presperse Inc.) and the C₇-C₈ through C₁₂-C₁₅ isoparaffins (such as the Isopart® Series available from Exxon Chemicals).

Non-polar, volatile oil may be included in the cosmetic composition to impart desirable aesthetic properties (e.g., good spreadability, non-greasy and/or tacky feel, quick drying to allow pigment particles to set on skin) to the present cosmetic composition. Non-polar, volatile oils suitable for use herein include silicone oils; hydrocarbons; and mixtures thereof. The non-polar, volatile oils may be either saturated or unsaturated, have an aliphatic character and be straight or branched chains or even contain alicyclic or aromatic rings. Examples of suitable non-polar, volatile hydrocarbons for use herein include polydecanes such as isododecane and isodecane (e.g., Permethyl-99A which is available from Presperse Inc.), dodecanes and tetra dodecanes (such as Parafol 12-97 and Parafol 14 from Sasol), and the C7-C8 through C12-C15 isoparaffins (such as the Isopar Series available from Exxon Chemicals). Exemplary non-polar, volatile liquid silicone oils are disclosed in U.S. Pat. No. 4,781,917. Additionally, a description of various volatile silicone oils may be found in Todd et al., “Volatile Silicone Fluids for Cosmetics”, Cosmetics and Toiletries, 91:27-32 (1976). Particularly suitable volatile silicone oils include cyclic volatile silicones corresponding to the formula:

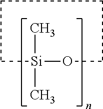

wherein n is from about 3 to about 7; and linear volatile silicones corresponding to Formula 1:

(CH₃)³Si—O—[Si(CH₃)²—O]^{m}—Si(CH₃)³)³ Formula 1

wherein m is from about 0 to about 7. Linear volatile silicone oils generally have a viscosity of less than about 5 centistokes at 25° C., whereas the cyclic silicones have viscosities of less than about 10 centistokes at 25° C. Examples of suitable volatile silicone oils include cyclomethicones of varying viscosities, e.g., Dow Corning 200, Dow Corning 245, available from Dow Corning Corp.); SF-1204 and SF-1202 Silicone Fluids (commercially available from Momentive Specialty Chemicals), and SWS-03314 (commercially available from Wacker Chemie AG.). In addition, Caprylyl Methicone such as Dow Corning FZ3196 can be used. Other examples of non-polar, volatile oils are disclosed, for example, in Cosmetics, Science, and Technology, Vol. 1, 27-104 edited by Balsam and Sagarin, 1972.

### Colorants

When the cosmetic composition is incorporated into products such as foundations, concealers, or mascaras, the cosmetic composition may include colorants. Colorants suitable for use in the present cosmetic compositions include, but are not limited to, dyes, pigments, lakes, and mixture thereof. (e.g., organic or inorganic pigments and colorants approved for use in eye-area cosmetics by PCPC and/or the FDA.) Exemplary inorganic pigments include particles of iron oxides (e.g., yellow, brown, red, black), titanium dioxides, iron sulfides, ultramarines, chromium oxides (e.g., green) or other conventional pigments used in cosmetic formulations. Examples of organic pigments include D&C Black No. 2. D&C Black No. 3, FD&C Red No. 40, D&C Green No. 5, FD&C Blue No. 1, and FD&C Yellow No. 5. Examples of lake dyes include various acid dyes which are laked with aluminum, calcium or barium. Additional colorants for use herein include annatto, caramel, carmine. ß-carotene, bismuth oxychloride, ferric ammonium ferrocyanide, ferric ferrocyanide, chromium hydroxides (e.g., green), guanine, mica, aluminum powder, bronze powder, copper powder, manganese violet, zinc oxide. Suitable colorants along with their chemical structure are described in, e.g., 21 C.F.R. Part 74 and in the PCPC Cosmetic Ingredient Handbook. (1988), published by the Cosmetics. Toiletry and Fragrances Association, Inc. Other colorants may also be used as they are developed and determined safe.

In one embodiment, cosmetic compositions according to the invention comprise from about 0.1 to about 70% by weight, for example from about 0.5 to about 50% by weight, and especially from about 1.0 to about 35% by weight based on the total weight of the composition, of a colorant. Colorants in the form of particles and/or encapsulants having average diameters of 0.1 to 50 microns may be acceptable for use in the present compositions. In another embodiment, the particles have average diameters of 0.1 to 10 microns. In another embodiment, the particles have average diameters of 0.1 to 5 microns. It may be desirable to select colorant particles with a diameter that is less than the thickness of the cosmetic composition dried-down film. The small size of the colorant particles may allow them to be fully encased in the dried film.

### Thickeners

When the cosmetic composition is incorporated into a formulation, the formulation may include thickeners. The composition can be thickened or structured with colloidal particles and/or waxes.

Thickening agents that may be useful in the present invention include carboxylic acid polymers such as the carbomers (e.g., the CARBOPOL® 900 series such as CARBOPOL® 954 by Lubrizol). Other suitable carboxylic acid polymeric agents include copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-30 alkyl acrylate crosspolymers and are commercially available as CARBOPOL® 1342. CARBOPOL® 1382, PEMULEN TR-1, and PEMULEN TR-2, from Lubrizol.

Additional suitable thickening agents include the polyacrylamide polymers and copolymers. An exemplary polyacrylamide polymer has the CTFA designation “polyacrylamide and isoparaffin and laureth-7” and is available under the trade name SEPIGEL 305 from Seppic Corporation (Fairfield. N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500 W, SSSA100H, from Lipo Chemicals, Inc., (Patterson. N.J.). Other suitable thickening agents useful herein are sulfonated polymers such as the CTFA designated sodium polyacryloyldimethyl taurate available under the trade name Simulgel 800 from Seppic Corp. and Viscolam At 100 P available from Lamberti S.P.A. (Gallarate, Italy). Another commercially available material comprising a sulfonated polymer is Sepiplus 400 available from Seppic Corp.

Waxes may be useful as thickeners and/or as structuring agents including natural synthetic, and surface modified waxes, including cold water process wax (such as CPW brands by JEEN International Corp). Waxes are defined as lower-melting organic mixtures or compounds of high molecular weight, solid at room temperature and generally similar in composition to fats and oils except that they contain no glycerides. Some are hydrocarbons, others are esters of fatty acids and alcohols. Waxes useful in the present invention are selected from the group consisting of animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof wherein the waxes have a melting point between 55° and 100° C. and a needle penetration value, as measured according to the American standard ASTM DS, of 3 to 40 units at 25° C. The principle of the measurement of the needle penetration according to the standards ASTM D5 consists in measuring the depth, expressed in tenths of a millimeter, to which a standard needle (weighing 2.5 g and placed in a needle holder weighing 47.5 g, i.e. a total of 50 g) penetrates when placed on the wax for 5 seconds. Waxes are used at levels in order to provide sufficient bulk material that resists drying out after application, providing thickness to the lashes.

Waxes may be useful to maintain the film durability of the composition. In some instances, the composition may include from 0.1-15% wax In another embodiment, the composition may include from 1-10% wax. In another embodiment, the composition may include from 4-8% wax. In some instances, it may be desirable to include wax at an amount of less than 3.0%, for example, less than about 1.0% or even less than 0.1%, by weight, of wax and wax-like components. In some instances, the present composition is free of wax.

Specific waxes that may be useful in the present invention include beeswax, lanolin wax, shellac wax (animal waxes): carnauba, candelilla, bayberry (vegetable waxes); ozokerite, ceresin, (mineral waxes): paraffin, microcrystalline waxes (petroleum waxes); polyethylene, (ethylenic polymers); polyethylene homopolymers (Fischer-Tropsch waxes); C₂₄₋₄₅ alkyl methicones (silicone waxes); and mixtures thereof. Most preferred are beeswax, lanolin wax, carnauba, candelilla, ozokerite, ceresin, paraffins, microcrystalline waxes, polyethylene, C₂₄₋₄₅ alkyl methicones, and mixtures thereof.

Clays may be useful to provide structure or thickening. Suitable clays can be selected, e.g., from montmorillonites, bentonites, hectorites, attapulgites, sepiolites, laponites, silicates and mixtures thereof. Suitable water dispersible clays include bentonite and hectorite (such as Bentone EW, LT from Rheox); magnesium aluminum silicate (such as Veegum from Vanderbilt Co.); attapulgite (such as Attasorb or Pharamasorb from Engelhard, Inc.); laponite and montmorillonite (such as Gelwhite from ECC America); and mixtures thereof.

Disteardimonium hectorite is a suitable thickener to build structure/viscosity in the present composition. For example, when it is used in a mascara formula this enables proper spreading/deposition across lashes, and ensures adequate stability/suspension of colorant particles in dispersion over time. It is preferable that the diameter of the disteardimonium hectorite is smaller than the thickness of the cosmetic composition dried-down film. The preferred diameter of the disteardimonium hectorite is less than 10 microns. The compositions may comprise from about 1% to about 25% of suitable thickener such as disteardimonium hectorite, from about 2% to about 20%, or even from about 3% to about 15%. Suitable thickening agents also include cellulose and modified cellulosic compositions such as, carboxymethyl cellulose, hydroxyethylcellulose, cellulose acetate propionate carboxylate, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers some portion of the hydroxy groups of the cellulose polymer are hydroyxalkylated (preferably hydroxyethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxvalkylcelluloses. Examples of alkyl groups useful herein include those selected from the group consisting of stearyl, isostearyl, lauryl, myristyl, cetyl, isocetyl, cocoyl (i.e. alkyl groups derived from the alcohols of coconut oil), pahnityl, oleyl, linoleyl, linolenyl, ricinoleyl, behenyl, and mixtures thereof. Preferred among the alkyl hydroxyalkyl cellulose ethers is the material given the PCPC designation cetyl hydroxyethylcellulose, which is the ether of cetyl alcohol and hydroxyethylcellulose. This material is sold under the tradename Natrosol® CS Plus from ASI.

### Actives

When the film forming composition is incorporated into a cosmetic formulation, the formulation may comprise a safe and effective amount of a biological, chemical, nutraceutical, or pharmaceutical active, or a combination thereof. Biological actives may include prostaglandins, antimicrobials, antibacterials, biocides, preservatives, proteins, amino acids, peptides, hormones, growth factors, enzymes (e.g., glutathione sulphydryl oxidase, transglutaminase), therapeutics, oligonucleotides, genetic materials (e.g., DNA, RNA), and combinations thereof. Chemical actives may include dyes, surfactants, sensates, hair conditioners, hair dyes, hair growth agents, hair styling gels, and combinations thereof. Nutraceutical actives may include proteins, preservatives, vitamins, food-additive materials, and combinations thereof. Pharmaceutical actives may include antibiotics, drugs, hair growth agents, and combinations thereof.

### Additional Polymers

In addition to the first and second copolymers, the composition may also include additional polymers.

The cosmetic composition of the present invention may comprise additional water-soluble film forming polymers. In one embodiment, water-soluble, film forming polymers comprise from about 1% to about 50%, preferably from about 2% to about 40% and most preferably from about 3% to about 30% of the composition.

The additional polymers comprise polymers formed from monomers, said monomer derivatives, mixtures of said monomers, mixtures of said monomer derivatives, natural polymers and mixtures thereof. The film forming polymers disclosed herein also include chemically modified versions of the above disclosed polymers. Said monomers are selected from the group consisting of olefin oxides, vinyl pyrrolidone, vinyl caprolactam, vinyl esters, vinyl alcohols, vinyl cyanides, oxazilines, carboxylic acids and esters and mixtures thereof. Preferred vinyl pyrrolidone polymers are selected from the group consisting of polyvinylpyrrolidone, vinyl acetate/vinyl pyrrolidone copolymer and mixtures thereof. Preferred polyvinyl esters are selected from the group consisting of vinyl acetate/crotonic acid copolymer, vinyl acetate crotonic acid vinyl neodecanoate copolymer and mixtures thereof. Preferred vinyl alcohol polymers are selected from the group consisting of vinyl alcohol vinyl acetate, vinyl alcohol/poly(alkyleneoxy)acrylate, vinyl alcohol/vinyl acetate/poly-(alkyleneoxy)acrylate and mixtures thereof. Preferred olefin oxides are selected from the group consisting of polyethylene oxide, polypropylene oxide and mixtures thereof. Preferred polycarboxylic acids and their esters are selected from the group consisting of acrylates, acrylates/octylacrylamide copolymers and mixtures thereof. The preferred oxaziline is polyoxaziline.

The additional polymers which may be useful in the present invention comprise natural polymers selected from the group consisting of cellulose derivatives, algin and its derivatives, starch and its derivatives, guar and its derivatives, shellac polymers and mixtures thereof. Preferred cellulose derivatives are selected from the group consisting of hydroxyethylcellulose, hydroxypropylcellulose, hydroxypropyl methylcellulose, ethylhydroxyethyl cellulose and mixtures thereof.

### Fats

Fats employed according to the invention are selected from the group consisting of fats derived from animals, vegetables, synthetically derived fats, and mixtures thereof wherein said fats have a melting point from about 55° C. to about 100° C. and a needle penetration value, as measured according to the American standard ASTM D5, from about 3 to about 40 units at 25° C. Preferably the fats selected for use in the present invention are fatty acid esters which are solids at room temperature and exhibit crystalline structure. Examples of fatty acid esters useful in the present invention include the glyceryl esters of higher fatty acids such as stearic and palmitic such as glyceryl monostearate, glyceryl distearate, glyceryl tristearate, palmitate esters of glycerol, C₁₈₋₃₆ triglycerides, glyceryl tribehenate and mixtures thereof.

### Plasticizing Solvents

Plasticizing solvents suitable for use herein are slow-evaporating, water-miscible or dispersible cosolvents that are 1) generally recognized as safe or 2) include slow evaporating glycols and glycol ethers, such as propylene glycol, butylene glycol; hexylene glycol; dipropylene glycol; dipropylene glycol methyl ether (commonly known as DPM); propylene glycol phenyl ether; and polyethylene glycols (PEGs) such as PEG 4 and PEG 8. Other exemplary plasticizing solvents include propylene carbonate, dimethyl isosorbide, and mixtures thereof. A wide variety of plasticizing solvents are listed in the CTFA International Cosmetic Ingredient Dictionary and Handbook, 3rd Ed., Cosmetic and Fragrance Assn., Inc., Washington D.C. (1982) pp. 575-580. The plasticizing solvent may be present in amounts of from 0.0% to 30% or even 5% to 20%, and generally appear in a ratio of solvent to polymer of from 10:1 to 1:5 or even 4:1 to 1:2. The plasticizing solvent is chosen to provide for water co-solvency, suitable solubility regarding the polymer, low volatility, stability, and safety (i.e., lack of toxicity). Thus, the cosmetic composition herein employs safe solvents that provide little or no sensation of tackiness or cooling (usually due to evaporation) on the applied area.

The plasticizing solvent may be chosen such that the polymer and plasticizing solvent are formulated in the aqueous phase of the emulsion, which may help reduce any tacky sensation of polymer contacting the user's hands and fingers during application of the cosmetic composition.

### Rheology Modifiers

Rheology modifiers that may be useful in the present invention include both associated and non-associated thickeners, including alkaline swellable, hydrophobic modified, polyurethane type thickeners and structuring agents. Useful rheology modifiers include natural gums and extracts, modified (semi-synthetic) gums and extracts, hydrophilic natural and synthetic silicate and clay mineral agents, hydrophobic silicas, inorganic and polymeric porous microparticle absorbents, synthetic polymers (such as acrylic polymers), and mixtures thereof.

Natural gums and extracts of the present invention are selected from, but not limited to, the group consisting of plant exudates, such as gum arabic, gum tragacanth, gum karaya, and gum ghatti; plant extracts, such as pectins; plant seed flours or extracts, such as locust bean gum, guar gun, psyllium seed gum, and quince seed gum; seaweed extracts, such as agar, alginates, and carrageenans: seed starches, such as corn starch, wheat starch, rice starch, and sorghum starch: tuber starches, such as tapioca starch and potato starch; animal extracts, such as gelatin and caseinates; and mixtures thereof.

Modified (semi-synthetic) gums and extracts of the present invention are selected from, but not limited to, the group consisting of cellulose derivatives, such as sodium carboxymethylcellulose, hydroxyethylcellulose, hydroxypropylcellulose, methylcellulose, and hydroxypropyl methylcellulose, as well as alkyl-modified cellulose derivatives, such as cetyl hydroxyethylcellulose; modified plant extracts, such as hydroxypropyl guar, microbial or biosynthetic gums, such as xanthan gum, sclerotium gum, gellan gum, dextran and its derivatives; modified starches and starch derivatives, such as potato starch modified, corn starch modified, hydroxypropyl starch, dextrin and its derivatives: modified animal derivatives, such as chitin or chitosan, and their derivatives, collagen derivatives; and mixtures thereof.

Hydrophilic natural and synthetic clay mineral agents of the present invention are selected from, but not limited to, the group consisting of hectorites, such as those sold under tradenames BENTONE® (Elementis Specialties); bentonites and montmorillonites, such as those sold under tradenames OPTIGEL®, GELWHITE® and MINERAL COLLOID® (by BYK Additives & Instruments), and POLARGEL® (AMCOL Health & Beauty Solutions): magnesium aluminum silicates, such as those sold under tradenames VEEGUM® (R. T. Vanderbilt Company), MAGNABRITE® (AMCOL Health & Beauty Solutions), and GELWHITE® MAS (BYK); sodium magnesium silicate, such as those sold under tradenames OPTIGEL® SH and LAPONITE® (both by BYK); lithium magnesium sodium silicate, such as LUCENTITE® SWN (Kobo Products); lithium magnesium silicate, such as LUCENTITE® SAN (Kobo Products); and mixtures thereof.

Hydrophobic silicas of the present invention are selected from, but not limited to, the group consisting of hydrophobically modified fumed silicas, such as WACKER HDK® H15, H20, and H30 (Wacker-Chemie), and hydrophobic grades under tradenames of AEROSIL® (Degussa AG) and CAB-O-SIL® (Cabot Corporation); and mixtures thereof.

Inorganic and polymeric porous microparticle absorbents of the present invention are selected from, but not limited to, the group consisting of high porosity/void volume fumed silicas, such as MSS-5003H and Silica Shells (both sold by Kobo Products), high porosity/void volume silicates like calcium silicate, such as sold under tradename HUBERDERM™ (J. M. Huber Corporation); high porosity/void volume polymeric particle absorbents including methacrylate polymers like allyl methacrylates copolymer, sold as POLY-PORE® E-200 (AMCOL Health & Beauty Solutions), and cross-linked dimethacrylate copolymers like lauryl methacrylateglycol dimethacrylate crosspolymer sold as POLYTRAP® 6603 (Enhanced Derm Technologies); high porosity cellulose beads like Cellulobeads® (Kobo Products); and mixtures thereof.

Synthetic polymers of the present invention include, but are not limited to, acrylic polymers, such as polyacrylates and polymethacrylates, and acrylic copolymers and crosspolymers, such as the carbomers or acrylates/C10-C30 alkyl acrylate crosspolymers sold under tradename CARBOPOL, (Lubrizol), and sodium polyacrylate sold under tradename RAPITHIX™ A-100 (ASI); alkali-soluble/swellable emulsion (ASE) polymers, hydrophobically-modified alkali-soluble/swellable emulsion (HASE) polymers, and hydrophobically-modified ethoxylated urethane (HEUR) polymers, such as those sold under tradename ACULYN™ (Dow Chemical Company) and STRUCTURE® (Akzo Nobel Company); hydrophobically-modified ethoxylate urethane alkali-soluble/swellable emulsion (HUERASE) polymers, such a those sold under tradename UCAR® POLYPHOBE® (Dow Chemical Company); copolymers of methyl vinyl ether and maleic anhydride, such as PVM/MA decadiene crosspolymer sold under tradename STABILEEZE® (ASI); hydrophobically modified non-ionic associative thickeners such as those sold under tradename PURE-THIX® (BYK); and mixtures thereof.

### Oil Soluble or Oil Dispersible Additives

The choice of oil-soluble or dispersible additive and the amount present according to the invention will depend on the intended use of the composition and the effectiveness of the compound. In top coat and remover compositions, the oil-soluble or dispersible additive chosen is acceptable for skin and eye contact, as is well known to the skilled formulator. Suitable oil-soluble or dispersible additives are incorporated at levels generally between 1 and 20% by weight based on the weight of the matrix bead (equivalent to 90 to 300% on weight of the colorant). Preferably 5 to 15% by weight of the oil-soluble or dispersible additive is employed.

The oil-soluble or dispersible additive may include fatty alcohols such as Guerbet alcohols based on fatty alcohols having from 6 to 30, preferably from 10 to 20 carbon atoms including lauryl alcohol, cetyl alcohol, stearyl alcohol, cetearyl alcohol, oleyl alcohol benzoates of C₁₂-C₁₅ alcohols, acetylated lanolin alcohol, etc. Especially suitable is stearyl alcohol. The oil-soluble or dispersible additive may include fatty acids such as Linear fatty acids of C₆-C₂₄, branched C₆-C₁₃ carboxylic acids, hydroxycarboxylic acids, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids). Further components that can be used are dicarboxylic acids of C₂-C₁₂, such as adipic acid, succinic acid, and maleic acid. Aromatic carboxylic acids, saturated and/or unsaturated, especially benzoic acid, can be used. Additional components that can be used as the oil soluble or dispersible additive include carboxylic acid salts; alkaline soaps of sodium, potassium and ammonium; metallic soaps of calcium or magnesium; organic basis soaps such as lauric, palmitic, stearic and oleic acid, etc., alkyl phosphates or phosphoric acid esters: acid phosphate, diethanolamine phosphate, potassium cetyl phosphate.

Other useful oil-soluble or dispersible additives comprise mild surfactants, super-fatting agents, consistency regulators, additional thickeners, polymers, stabilizers, biologically active ingredients, deodorizing active ingredients, anti-dandruff agents, film formers, swelling agents, UV light-protective factors, antioxidants, preservatives, insect repellents, solubilizers, colorants, bacteria-inhibiting agents, hair conditioning agents, vitamins, and the like.

### Test Methods

### Contraction Test 1

The principle of the contraction measurement is based on the shrinkage degree of a substrate after polymer compositions are applied to substrate and dried. The method is specifically used for a polymer system with higher contraction modulus when a substrate with high elastic modulus is to be tested or predicted, such as eye lashes.

### Equipment:

1. Leneta cards Form 2A (double coated opacity) with a dimension of 14 cm×25.4 cm, supplied by Leneta Company.
2. Single bar 3-inch Film Applicator, 6 mils thickness, supplied by BYK Gardner
3. Drawdown Plate PA4200, supplied by BYK Gardner
4. Digital Humidity/Temperature Meter (Traceable® Model 35519-044 from VWR), or equivalent
5. Digital balance (with minimum sensitivity of at least 0.001 g)
6. Measuring ruler (30 cm with mm scaling)

### Procedure:

1. Pre-weigh the Leneta card and record weight before a drawdown process. The drawdown method may refer ASTM D4062 or ASTM D2805 standard test methods.
2. Position and secure the Leneta card on the Drawdown Plate.
3. Place the bar film applicator centered at the top of the card, and load 5-10 grams of polymer compositions distributed evenly across and immediately in front of the bar applicator. Ensure the amount of polymer composition load is sufficient to pass over the end of the card that gives a covered area of about 3 inches×8 inches by the casted film.
4. Drawdown uniformly in the center of the card all the way down, and pass the polymer compositions to the end of the card and onto the drawdown plate. Ensure the casted film is evenly distributed and in an essentially rectangular shape.
5. Allow the film to dry in a horizontal position for minimum 4 hrs (typically overnight).
6. Conduct experiments at a relative humidity of 40-50% and at a temperature in the range of 22° C. to 28° C.
7. After the film is dried, the card is weighed again to determine the amount of total solids of the polymer compositions loaded by calculating the differences between the weights before and after the film cast.

### Measurement and Calculation:

Depending on the type of contraction or curling effect observed for a given composition on a card, only one of the calculation formulas below should be selected for best evaluations of the contraction under specified relative humidity and temperature conditions.

1. On a flat, untreated Leneta card (such as shown in FIG. 1), measure the distances (measured to nearest tenth of cm) from the top edge to bottom edge of the card in both right (R) and left (L) sides of the card. As shown in FIG. 1. L is the length on the left side of the card and R is the length on the right side of the card. Then, for an evenly contracted card (such as shown in FIG. 2), measure the L and R lengths after treatment with a polymer composition and allowing the card to dry. The degree of contraction (% Contraction) of the polymer composition is calculated per Formula 2:

% Contraction=100×[1−(R+L)/(25.4×2)] Formula 2

For example:

If L=7.1 cm, and R=6.9 cm of an evenly contracted card (such as shown in FIG. 2), the % contraction is calculated per Formula 3:

% Contraction=100×[1−(6.9+7.1)/(25.4×2)]=72.4% Formula 3

2. For non-even contraction or twisted cards, such as shown in FIG. 3, measure the distances (measured to nearest tenth of cm) from the top edge to bottom edge of the card in both right (R) and left (L) sides of the card, and also the distances of the diagonal of the card from the right top to left bottom (RL) and from the left top to right bottom (LR). The degree of contraction (% Contraction) of the polymer composition is calculated per Formula 4:

% Contraction=100×[1−(R+L+RL+LR)/(25.4×2+29×2)] Formula 4

For example, if L=5.8 cm, R=2.9 cm, LR=13.6, and RL=15.2 of a non-evenly contracted twisted card, the % Contraction is calculated per Formula 5:

% Contraction=100×[1−(5.8+2.9+13.6+15.2)/(25.4×2+29×2)]=65.5% Formula 5

3. For coiled cards (such as shown in FIG. 4), measure diameters on both right (dR) and left (dL) edges (measure to nearest tenth of cm). The degree of contraction (% Contraction) of the card is calculated per Formula 6:

% Contraction=100×3.4218+(1/dR+1/dL) Formula 6

For example, if dL=7.2 cm and dR=6.3 cm of a coiled card (where dL and dR are diameters measured from left side and right side respectively), the % Contraction is calculated per Formula 7:

% Contraction=100×3.4218×(1/7.2+1/6.3)=102% Formula 7

The Leneta card Contraction Test is conducted primarily for polymer technical screening. The Leneta card method can be used in combinations with other contraction methods for contraction performances of polymer systems. Other methods may include image analysis method for curling and eyelash/hair lifting on false lashes, image analysis method on human eye lashes, and consumer panel test.

### Contraction Test 2

The principle of the contraction measurement is based on the shrinkage degree of a substrate after polymer compositions are applied to substrate and dried. The method is specifically used for a polymer system with weaker contraction modulus when a substrate with lower elastic modulus is to be tested or predicted, such as human skins.

### Equipment:

1. Form WNT-34—Drawdown Sheets from Leneta Company
2. Lcncta drawdown plates (clipboard) from Leneta Company
3. Coating rods RDS-2, RDS-3, and RDS-6 from R. D. Specialties
4. Disposable DB Latex-free plastic syringes (5 ml) from VWR
5. Sanitizing aids—DI water and Isopropyl alcohol (IPA), Paper towel
6. Polymer samples at concentrations of 1%-20%

### Procedure

1. Clean the working area where tests to be performed.
2. Clean the coating rods, drawdown plate, and any other tools with DI water, then sanitize again with IPA.
3. Label Leneta sheets with date, formula/batch #, applicator thickness, volume of product, and % relative humidity (RH).
4. Pre-weigh the labeled sheets and record the weight.
5. Confirm the length 1 (19.3 cm), width w (12.7 cm) and diagonal d (23.1 cm) of Leneta sheets.
6. Secure Leneta sheet onto clipboard. Cover the least amount of paper as possible to maximize the available testing space.
7. Place applicator against the clip at the top of the Leneta sheet The applicator should not cover the label.
8. Add 6 mL of product at the top of the sheet using the plastic syringe. Apply evenly making sure that no product spills from the sheet.
9. Keeping even pressure, roll the applicator straight down past the end of the Leneta sheet and onto the glass plate. Ensure that all or most of the sheet is evenly covered with the product.
10. Remove the Leneta sheet from the clipboard and place it onto a flat benchtop surface. Be sure to guide the curl of the sheet in the proper direction using gloves. Excessively curling the sheet can result in bending the paper or artificial curl.
11. Day the coated sheets for more than 4 hours (typically overnight).
12. Measure, in centimeters, the distance from the top left corner to the bottom left corner (L). Repeat for top right corner to bottom right corner (R).
13. Measure, in centimeters, the distance from the top left corner to the bottom right corner (LR). Repeat for top right corner to bottom left corner (RL). These values are used when dried sheets are twisted.
14. Weigh the coated Leneta sheets after the sheets are dry, and record.

### Measurements and Calculations

1. Formula 8 was used to calculate uniform contraction of Leneta sheets:

$$\% Shrinkage = 100 * ( 1 - \frac{L + R}{2 l} ) \qquad Formula 8$$

2. Formula 9 was used to calculate twisted or non-uniform shrinkage of Leneta sheets:

$$\% Shrinkage = 100 * ( 1 - \frac{L + R + LR + RL}{2 l + 2 w} ) \qquad Formula 9$$

3. Formula 10 was used to calculate fully curled Leneta sheets (in a circle) using curvature (k):

$$k = 100 * ( \frac{6.1434}{d} ) \qquad Formula 10$$

D=average of diameters measured in vertical and horizontal directions for both sites of the Leneta sheet The WNT-34 Leneta sheet is 12.7 inches wide and 19.3 inches long.

### Operation Recommendations

1. 6 mil film thickness (wet) is recommended for general evaluations, specifically for dilute or less contractible polymer solutions.
2. For more concentrated polymer solutions (eg >10%), 3 mil film thickness (wet) is recommended.
3. No significant differences observed in 3 mil and 2 mil roller casting, and too much variation for 2 mil roller at more dilution or less contractible polymer solutions.

### Weight Loss Test (for Dry Speed Evaluation)

The principle of the Dry Speed measurement is based on weight loss of the polymer compositions due to evaporation of volatile components (carrier/solvents) with time under specified conditions of relative humidity (% RH) and temperature.

### Equipment:

1. Digital balance (Model AT460 4 decimal digits by Mettler Toledo with balance chamber enclosure), or equivalent
2. Air flow meter (Kontes by Granger), or equivalent
3. Dry nitrogen gas supply (Compressed) by Air Gas
4. Glass microscope slides (3-inch×1-inch×1-mm) from VWR
5. ⅞ inch hole Arch Punch
6. Hammer
7. Films: Bytac® type VF-81/FEP PTFE protection film (9 mil thickness) available from Saint Gobain-Performance Plastics (Item#1435-AB)
8. Straight-edged scraper (Precision Gate & Tool A-1)
9. Digital timer

### Procedure:

1) Prepare film template strips by cutting the Bytac protection films into about 1.5 inches×2.5 inches size.

2) Punch a ⅞ inch hole centered at one end of the Bytac strip using the Arch Punch and hammer.

3) Turn on Nitrogen gas, and set automatic stop with defined time (usually about 2 hours).

4) Check and make sure gauge attached to balance chamber with flow meter reading at 1.1 liter per min.

5) Put a glass slide into the balance chamber and tare the glass slide weight on the balance, then remove glass slide from the balance.

6) Remove the protective layer from the back side of the Bytac film template and attach it evenly and carefully onto the glass slide with the hole positioned at the middle of the slide.

7) Press the film template with a clean straight-edged scraper up and down to remove air trapped under the film.

8) Load about 1 g of a polymer sample onto the top side of the hole.

9) Draw the polymer sample evenly across the hole on the glass slide using the straight-edged scraper to cover the hole area completely.

10) As soon as product is applied, peel the film template off the slide.

11) Immediately put the glass slide with product back to the balance, close the balance chamber door and start timing.

12) Record relative humidity (% RH) and temperature of the test condition.

13) The weight loss test will be automatically stopped when the weight loss reaches equilibrium if the balance is interfaced with a computer, or manually stopped when the recorded weight is no longer changed.

14) Record the weight changes for every 15-20 seconds until the weight loss has reached the equilibrium or minimum via any suitable computer software program or by manual recording.

15) Generate a drying profile graph of weight of the polymer composition against the time (in seconds) during the whole drying process based on the record.

### Measurement and Calculation:

Based on the drying profile, determine 90/o dry weight (as 90% total weight loss) and corresponding time.

90% dry weight=Starting Sample Wt.−0.9×[Starting Sample Wt.−Final Sample Wt. (at 60 minutes drying or at drying equilibrium)]

90% dry time=corresponding time (in minutes) taken to reach 90%/o dry weight

The Dry Speed of a polymer composition is defined as the 90% dry time, or time required to reach 90% total weight loss.

### Examples

Particle types, physical properties, specifically surface hydrophilicity, particle sizes and specific surface areas can impact the performance of the tensioning polymer compositions, specifically in film contractions and film dry time. The data presented below shows that precipitated silica particles outperform other types of silica particles (including silica microsphere, fumed silica and fused silica) under test conditions in a composition with the tensioning polymer system.

Table 1 shows the performance of selective silica particles. Among the tested silica particles, precipitated silica Sipernat 50S delivers improved performance benefits of the tensioning polymer system in both film contraction and film drying speed, while other types of particles have negative impacts on film contraction at the equal particle/polymer ratio.

TABLE 1
Performances of different types of silica particles in a polymer composition

| | | | | | Pore | | |
|---|---|---|---|---|---|---|---|
| | % Shrinkage | 90% dry | D50 | SSA | volume | | surface |
| | (44-47% RH) | (min) | (μ) | (M2/g) | (ml/g) | Structure | property |
| without silica | 16.51 | 12.51 | | | | | |
| Sipernat50S | 22.39 | 7.93 | 16 | 475 | | Precipitated | Hydrophilic |
| | | | | | | silica | |
| Silica Shells | 1.56 | 10.68 | 3 | 120 | 0.2 | Silica | Hydrophilic |
| | | | | | | microsphere | |
| Silica Shells-SH | 1.93 | 6.41 | 3 | | | Silica | Hydrophobic |
| | | | | | | microsphere | |
| MSS-500/3N | 0.55 | 28.37 | 3 | 40 | 0.05 | Silica | Hydrophilic |
| | | | | | | microsphere | |
| MSS-500/3 | 13.21 | 10.37 | 3 | 800 | 1 | Silica | Hydrophilic |
| | | | | | | microsphere | |
| MSS-500/3H | 0.92 | 8.85 | 3 | 700 | 2 | Silica | Hydrophilic |
| | | | | | | microsphere | |
| Aerosuk R805 | 12.39 | 7.93 | 12 | 150 | | Fumed silica | Hydrophobic |
| Teco SphereA | 3.44 | 10.68 | 15 | 0.92 | | Fused silica | Hydrophilic |
| Teco Sphere | 0.41 | 9.46 | 22.5 | 0.75 | | Fused silica | Hydrophilic |

Base Composition for Table 1: 20% Polymer system+10% Ethanol+5% particles+water QS Silica particles with hydrophilic surface property outperform hydrophobically modified silica particles in an aqueous phase of the polymer compositions. Table 2-a shows the impact of the surface hydrophilicity of selected precipitated silica particles on the performances of a tensioning polymer composition.

TABLE 2-a
Impact of surface hydrophilicity of precipitated silica particles

| | | | | | Pore | | |
|---|---|---|---|---|---|---|---|
| | % Shrinkage | 90% dry | D50 | SSA | volume | | surface |
| | (44-47% RH) | (min) | (μ) | (M2/g) | (ml/g) | Structure | property |
| without silica | 16.51 | 12.51 | | | | | |
| Sipernat50S | 22.39 | 7.93 | 16 | 475 | | Precipitated | Hydrophilic |
| | | | | | | silica | |
| Spheron LC-KAA | 12.2 | 14.64 | 5 | 215 | 0.8 | Precipitated | Hydrophilic |
| | | | | | | silica | |
| SpheronP1500 | 0.32 | 7.02 | 5 | | 0.3 | Precipitated | Hydrophobic |
| | | | | | | silica | |

Base Composition for Table 2-a: 20% Polymer system+10% Ethanol+5% particles+water QS Table 2-b shows the impact of the surface hydrophilicity of selected silica microsphere particles on the performances of a tensioning polymer composition with the same particle sizes.

TABLE 2-b
Impact of surface hydrophilicity of silica microsphere particles

| | | | | | Pore | | |
|---|---|---|---|---|---|---|---|
| | % Shrinkage | 90% dry | D50 | SSA | volume | | surface |
| | (44-47% RH) | (min) | (μ) | (M2/g) | (ml/g) | Structure | property |
| Silica Shells | 1.56 | 10.68 | 3 | 120 | 0.2 | Silica | Hydrophilic |
| | | | | | | microsphere | |
| MSS-500/3N | 0.55 | 28.37 | 3 | 40 | 0.05 | Silica | Hydrophilic |
| | | | | | | microsphere | |
| MSS-500/3 | 13.21 | 10.37 | 3 | 800 | 1 | Silica | Hydrophilic |
| | | | | | | microsphere | |
| MSS-500/3H | 0.92 | 8.85 | 3 | 700 | 2 | Silica | Hydrophilic |
| | | | | | | microsphere | |
| Silica Shells-SH | 1.93 | 6.41 | 3 | | | Silica | Hydrophobic |
| | | | | | | microsphere | |

Base Composition for Table 2-b: 20% Polymer system+10% Ethanol+5% particles+water QS

The film contraction performance of the tensioning polymer systems was found to increase significantly with reduced particle sizes with the same or similar physical properties in surface areas. As expected, smaller particles are more effective in film reinforcement due to more available surface areas for polymer-particle interactions (bridging effects).

Table 3 shows the impact of the median particle size (D50) of the leading silica particles (with the same surface area) on contraction performances of the tensioning polymer system.

TABLE 3
Impacts of particle sizes of silica particles

| | % Shrinkage | 90% dry | D50 | SSA | | surface |
|---|---|---|---|---|---|---|
| | (30-34% RH) | (min) | (μ) | (M2/g) | Structure | property |
| Sipernat50S | 37 | 2.4 | 16 | 475 | Precipitated | Hydrophilic |
| | | | | | silica | |
| Sipernat500LS | 47 | 3.1 | 6 | 475 | Precipitated | Hydrophilic |
| | | | | | silica | |

Base Composition for Table 3: 20% Polymer system+10% Ethanol+5% particles+water QS (120926)

The film contraction and film dry performance of the tensioning polymer system is better with increased surface contact areas (higher surface area and lower pore volume). The surface areas of outer layers of particles are expected to play a more important role due to higher polymer-particle connections. On the other hand, inner pore surface areas enhance the dry speed of the polymer system.

Table 4-a shows the enhanced film contractions and dry speed of the tensioning polymer system by increasing the surface areas of silica microsphere particles with similar particle sizes and hydrophilicity. An exception is MSS-500/3H that has a high surface area but led to weak film contraction. Without being bound by theory, this is likely related to lower exposable surface areas, as expected by its high internal pore volume (2 ml/g).

Table 4-a also shows the impact of pore volume of silica microsphere particles on performances of the tensioning polymer system at the same particle surface hydrophilicity.

TABLE 4-a
Impact of surface areas of silica microsphere particles

| | | | | | Pore | | |
|---|---|---|---|---|---|---|---|
| | % Shrinkage | 90% dry | D50 | SSA | volume | | surface |
| | (44.47% RH) | (min) | (μ) | (M2/g) | (ml/g) | Structure | property |
| MSS-500/3H | 0.92 | 8.85 | 3 | 700 | 2 | Silica | Hydrophilic |
| | | | | | | microsphere | |
| MSS-500/3 | 13.21 | 10.37 | 3 | 800 | 1 | Silica | Hydrophilic |
| | | | | | | microsphere | |
| Silica Shells | 1.56 | 10.68 | 3 | 120 | 0.2 | Silica | Hydrophilic |
| | | | | | | microsphere | |
| MSS-500/3N | 0.55 | 28.37 | 3 | 40 | 0.05 | Silica | Hydrophilic |
| | | | | | | microsphere | |

Base Composition for Table 4-a: 20% Polymer system+10% Ethanol+5% particles+water QS

Table-4-b shows the contraction performances of the tensioning polymer films affected by the surface area of silica particles with variations in particle types, surface properties and particle sizes. Higher surface areas (SSA) in general lead to higher contractions, even with larger particle sizes or hydrophobic particles.

Without being bound by theory, our hypothesis is that shrinkage is more correlated to surface areas that are more exposed to polymers where polymer-particle interactions led to reinforced polymer interpenetrations and network.

TABLE 4-b
Impact of surface areas of various silica particles

| | | | | | Pore | | |
|---|---|---|---|---|---|---|---|
| | % Shrinkage | 90% dry | D50 | SSA | volume | | surface |
| | (44-47% RH) | (min) | (μ) | (M2/g) | (ml/g) | Structure | property |
| Sipernat50S | 22.39 | 7.93 | 16 | 475 | | Precipitated | Hydrophilic |
| | | | | | | silica | |
| Spheron LC-KAA | 12.2 | 14.64 | 5 | 215 | 0.8 | Precipitated | Hydrophilic |
| | | | | | | silica | |
| Aerosuk R805 | 12.39 | 7.93 | 12 | 150 | | Fumed silica | Hydrophobic |
| Teco SphereA | 3.44 | 10.68 | 15 | 0.92 | | Fused silica | Hydrophilic |
| Teco Sphere | 0.41 | 9.46 | 22.5 | 0.75 | | Fused silica | Hydrophilic |
| SpheronP1500 | 0.32 | 7.02 | 5 | | 0.3 | Precipitated | Hydrophobic |
| | | | | | | silica | |

Base Composition for Table 4-b: 20% Polymer system+10% Ethanol+5% particles+water QS

Film dry time of the tensioning polymer system is impacted by two factors—surface hydrophobicity of silica particles (regardless other physical properties) and pore volume of the same types of silica particles.

Table 5 shows the impact of the surface hydrophobicity of various silica particles on polymer performances.

TABLE 5
Impacts of surface hydrophobicity of various silica particles

| | | | | | Pore | | |
|---|---|---|---|---|---|---|---|
| | % Shrinkage | 90% dry | D50 | SSA | volume | | surface |
| | (44-47% RH) | (min) | (μ) | (M2/g) | (ml/g) | Structure | propcity |
| Silica Shells-SH | 1.93 | 6.41 | 3 | | | Silica | Hydrophobic |
| | | | | | | microsphere | |
| SpheronP1500 | 0.32 | 7.02. | 5 | | 0.3 | Precipitated | Hydrophobic |
| | | | | | | silica | |
| Aerosuk R805 | 12.39 | 7.93 | 12 | 150 | | Fumed silica | Hydrophobic |
| Sipernat50S | 22.39 | 7.93 | 16 | 475 | | Precipitated | Hydrophilic |
| | | | | | | silica | |
| MSS-500/3H | 0.92 | 8.85 | 3 | 700 | 2 | Silica | Hydrophilic |
| | | | | | | microsphere | |
| Teco Sphere | 0.41 | 9.46 | 22.5 | 0.75 | | Fused silica | Hydrophilic |
| MSS-500/3 | 13.21 | 10.37 | 3 | 800 | 1 | Silica | Hydrophilic |
| | | | | | | microsphere | |
| Silica Shells | 1.56 | 10.68 | 3 | 120 | 0.2 | Silica | Hydrophilic |
| | | | | | | microsphere | |
| Teco SphereA | 3.44 | 10.68 | 15 | 0.92 | | Fused silica | Hydrophilic |
| Spheron LC-KAA | 12.2 | 14.64 | 5 | 215 | 0.8 | Precipitated | Hydrophilic |
| | | | | | | silica | |
| MSS-500/3N | 0.55 | 28.37 | 3 | 40 | 0.05 | Silica | Hydrophilic |
| | | | | | | microsphere | |

Blase Composition or Table 5: 20% Polymer system+10% Ethanol+3% particles+water QS

The data showed improved performance from precipitated silica. Sipernat50S and Sipernat500LS silica delivered significant contraction and film set/dry benefits for the tensioning polymer system. Notable parameters of the lead particles include their surface hydrophilicity and external surface areas of particle (related specific surface areas and pore volumes).

Most of the particles tested showed negative effects on the polymer performances (shrinkage and film set). The particles tested in this work include silica microspheres, precipitated silica, fused silica, fumed silica, and organic (Nylon particles) with variations in physical properties (structure types, surface hydrophilicity, particle sizes, and surface areas, and pore volumes). Surprisingly, precipitated silicas Sipernat 500LS and 50LS were found to deliver positive effects on the performances of the tensioning polymers (higher shrinkage and shorter film set time).

Film Dry:

Most porous and hydrophobic particles in the study showed benefits in film formation with fast drying times, likely driven by capillary effects and polymer interactions on the particle surfaces. In contrast, fused/non-porous silica and fumed silica thickeners showed negative effects, likely due to weak polymer-particle interactions and weak capillary effects/slow solvent evaporation.

Shrinkage:

All tested fillers showed more or less reduced shrinkage benefits, except precipitated hydrophilic silica. Without being bound by theory, we speculate that precipitated hydrophilic silica leads to strengthened polymer-polymer network formation as such formation is reinforced by particle-polymer interactions on particle surfaces.

Viscosity:

Solid contents lead to raised viscosity. Precipitated fillers resulted in relatively less increase in viscosity, while fumed silica fillers led to more significant viscosity increases.

### Skin Tensioning Test

The DermaTOP 2D/3D scanning system provides fast analysis of surface characteristics on human skin (in-vivo) and skin mimic substrates (in-vitro). A special blue light source imparts high contrast when projected on textured surfaces. Dermatop HE-50 with Trianglulation angle 30 degree. Field of view 40×30×20 mm3, Resolution 35 micron in XY and 4 micron in Z dimension was in this study. Fifty (50) data points were generated at each evaluation area. The imaging system includes highly automated software for quick and easy analysis. Roughness parameters (Ra and Rz) are available for direct assessment of product target areas.

Based on optical triangulation, the DermaTOP uses the fringe projection technique to capture a 3D surface topography map. In this study, it was configured to capture the topography of the face skin, including forehead, crow's feet, cheeks near the eyes, and smile lines before and after product application. The DermaTop data is used to measure the texture modification effects of the products or formulations, including fine line reduction, pore minimizations, and anti-wrinkle effects.

Measurement and Calculation:

Two parameters were used to evaluate surface roughness. Lower roughness indicates more smoothness of the surfaces.
1. Ra—average wrinkle depth
2. Rz—maximum wrinkle depth
3. % Change before and after (higher % means more effective in texture modification)

% Change=(roughness data baseline−roughness data after product application)/roughness data after product application

Skin Tensioning Test Results

Panelists were washed with Olay face cleaning product, and wait for 10 min before the test. After 10 min rest, the bare face of the panelists were imaged by DermaTOP, and the baseline data was collected. A polymer was applied onto the face using finger. After resting for 3 minutes (allow the product to dry), the final DermaTOP data was collected.

TABLE 6
DermaTop roughness data

| | |
|---|---|
| Forehead | Crow's feet |

| | Ra (mm) | Rz (mm) | Ra (mm) | Rz (mm) |
|---|---|---|---|---|
| Bare | 0.04977 | 0.13317 | 0.04297 | 0.11345 |
| Applied | 0.01371 | 0.04438 | 0.01837 | 0.04205 |
| % change | 263% | 200% | 134% | 170% |

Base Formulation for Table 6: Polymer (5% Advantage S and 5% Aquastyle 300), Particle (2% Sipernat 500LS), and 3% Ethanol

### Pigmented Compositions—Lipophilic Pigments

Pigment types, surface properties (Lipophilicity/surface treatment), and particle dose (both pigment and filler levels) can play important roles in the contraction behaviors of tensioning compositions. Lipophilic pigment particles improve the contraction performance of an un-pigmented polymer composition. The addition of hydrophilic pigments (e.g. untreated silica) and stabilizers (e.g. glycerin) to the polymer composition led to reduced polymer film contractions. Levels of lipophilic pigments and hydrophilic fillers in the pigmented polymer compositions can both impact the film contractions.

TABLE 7
Contraction performances of silica particle in combinations with pigment particles

| | | Kobo Pigment | Sensient-EM | Sensient-AS | Sensient-AS | Sensient-AS |
|---|---|---|---|---|---|---|
| | No pigment | slurry | (hydrophilic) | (lipophilic) | (lipophilic) | (lipophilic) |
| 500LS silica | 5.0% | 5.0% | 5.0% | 5.0% | 5.0% | 3.0% |
| Pigment | 0.0% | 3.6% | 6.0% | 6.0% | 3.6% | 6.0% |
| pH | 7.68 | 7.70 | 7.48 | 7.37 | 7.51 | 7.55 |
| Viscosity (Pa-s) | 36.0 | 56.0 | 98.4 | 108.0 | 44.8 | 57.6 |
| Shrink RH37% | 47.5% | 37.6% | 60.6% | 80.9% | 52.1% | 42.7% |

Base Composition for Table 7-20% Polymers+10% Ethanol+5% Sipernat500LS+0.1% MethylParaben+water QS)

A pigment was identified for the present tensioning polymer composition that is a lipophobically treated pigment (Covalumine Sonoma AS).

Table 8 lists the contraction performances of iron oxide pigments with different surface properties (polarity, particle size and particle size distribution defined by D90/D50). Lipophilic pigments outperform the hydrophilic ones. Among the similar surface properties, pigments with smaller panicle size tend to perform better in general based on filler reinforcement theory.

TABLE 8
Contraction performances of lipophilic vs hydrophilic pigments in a polymer
composition containing a hydrophilic filler particle (Sipernat500LS)

| | Vis | | Shrinkage % | PS | | | |
|---|---|---|---|---|---|---|---|
| Iron Oxide | (Pa-s) | pH | (28-37% RH) | (μ) | D90/D50 | Property | Vendor |
| GLW60GBSP | 36 | 7.7 | 39.9% | 1.60 | 1.50 | Dispersion w/glycerin | Kobo |
| | | | | | | (hydrophilic) | |
| W60GBSP | 56 | 7.3 | 46.0% | 1.60 | 1.50 | Dry powder | Kobo |
| | | | | | | (hydrophilic) | |
| Unipure-EM | 98.4 | 7.4 | 48.2% | 2.00 | 1.90 | Treated w/silica | Sensient |
| | | | | | | (hydrophilic) | |
| Covalumine | 70 | 7.2 | 80.9% | 9.00 | 1.57 | Triethoxycaprylylsilane | Sensient |
| Sonoma AS | | | | | | coated on Al (lipophilic) | |
| C33-5198 | 94 | 7.3 | 58.8% | 0.55 | 0.55 | Synthetic/no surface | SunChemical |
| | | | | | | treatment (hydrophilic) | |
| C33-5000 | 98 | 7.4 | 51.7% | 0.65 | 0.81 | Synthetic/no surface | SunChemical |
| | | | | | | treatment (hydrophilic) | |
| Sympholight | 52 | 7.7 | 71.6% | 0.40 | | Silica surface bonded | Presperse |
| BW -TE | | | | | | (hydro/lipophilic) | |
| Aquaspersabil | 96.0 | 7.10 | 22.0% | 1.38 | 1.93 | C14-16 sulphonate | Presperse |
| BKIO | | | | | | surface treated | |
| | | | | | | (hydrophilic) | |
| Bismica Max | 50.0 | 7.00 | 34.3% | 5.98 | 2.39 | Mica/BiOCl loaded | Presperse |
| | | | | | | (hydrophilic) | |

Base Formula for Table 8: 20% Polymers+10% Ethanol+5% Sipernat500LS+0.1% MethylParaben+6% various Pigment+water QS)

Table 9 demonstrates that silica filler particles can have significant impact on the contraction and surface texture of a pigmented polymer system. Sipernat500LS reached shrinkage plateau at around 5% (close to CPWC), but significant cracking at and above 5% that led to poor surface appearance (entry 6. Table 9), due to insufficient dry film flexibility (similar to the un-pigmented system). Sipernat D13 in a combination with 500LS (@5%) improves film smoothness (similar to the un-pigmented system). Roughly equal level of D13 to 500LS silica was required to deliver smooth film surface of pigmented polymer system, specifically at higher levels of 500LS (entries 6-9 cracked vs 10-11 smooth surfaces, Table 9). Different from un-pigmented system, Sipernat D13 silica alone showed improved contraction vs without silica with a maximal contraction at −5% (likely close to CPWC) without surface smoothness reductions in the pigmented system (entry-5 vs entry-1, Table 9). Although the maximal contraction was lower than that of Sipernat500LS.

TABLE 9
Impacts of silica particles on Contraction and film
surface of pigmented aqueous polymer system

| | Total | Sonoma | Total | Sipernat | Sipernat | % | |
|---|---|---|---|---|---|---|---|
| | particles | AS | silica | D13 | 500LS | Contraction | Film surface |
| 1 | 6 | 6 | 0 | 0 | 0 | 32% | Smooth |
| 2 | 8 | 6 | 2 | 0 | 2 | 69% | Smooth |
| 3 | 11 | 6 | 5 | 3 | 2 | 86% | Smooth |
| 4 | 11 | 6 | 5 | 2 | 3 | 127% | Smooth |
| 5 | 11 | 6 | 5 | 5 | 0 | 133% | Smooth |
| 6 | 11 | 6 | 5 | 0 | 5 | 187% | 100% peeling + |
| | | | | | | | significant cracking |
| 7 | 12 | 6 | 6 | 1 | 5 | 105% | 100% Peeling + |
| | | | | | | | cracking |
| 8 | 14 | 6 | 8 | 3 | 5 | 128% | 100% peeling + slight |
| | | | | | | | cracking |
| 9 | 16 | 6 | 10 | 0 | 10 | 21% | 100% peeling + |
| | | | | | | | significant cracking |
| 10 | 16 | 6 | 10 | 5 | 5 | 72% | Smooth |
| 11 | 17 | 6 | 11 | 6 | 5 | 31% | Smooth |

Base Formula for Table 9: 20% polymers+10% Ethanol+1% CPW-B+6% Covalumine Sonoma AS+various % Sipernat500LS+various % Sipernat D13+water QS)

In the presence of 5% Sipernat500LS, D13 silica showed maximal contraction at 3% suggesting possible CPWC at near 3% in D13 silica and of 8% in total particles (entry 8. Table 9) in a mixed suspension with pigment (6% Sonoma AS) and 500LS silica (5%).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Additionally, properties described herein may include one or more ranges of values. It is to be understood that these ranges include every) value within the range, even though the individual values in the range may not be expressly disclosed.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A cosmetic composition having an aqueous phase, the aqueous phase comprising:
a. A film-forming tensioning polymer system comprising:
i. a first non-crosslinking polyamide/polyacrylate copolymer comprising the following monomer units:
i. at least one amide monomer, including vinyl caprolactam monomers, vinylpyrrolidone monomers, and acrylamide monomers;
ii. (meth)acrylate monomers;
iii. monomers having at least one carboxylic functional group selected from the group consisting of carboxylic esters, carboxylic acids, their salts, or precursors of carboxylate functions, and mixtures thereof; and
iv. monomers having at least one amine functional group including primary, secondary and tertiary amines
ii. a second non-crosslinking polyamide copolymer comprising the following monomer units:
i. at least one amide monomer, including vinyl caprolactam monomers, vinylpyrrolidone monomers, and (meth)acrylamide monomers;
ii. at least one quaternary ammonium containing monomer and
iii. monomers having at least one amine functional group including primary, secondary, and tertiary amines; and
B. at least one hydrophilic non-colloidal particulate agent comprising precipitated silica particles, lipophilically treated pigment powders, hydrophobically treated pigment powders, or combinations thereof.

2. The cosmetic composition of claim 1 wherein said film-forming tensioning polymer system is adhesive.

3. The cosmetic composition of claim 1 wherein said first non-crosslinking polyamide/polyacrylate copolymer comprises a polyvinylcaprolactam/vinylpyrrolidone/dimethylaminoalkyl methacrylate copolymer; and further, wherein said first non-crosslinking polyamide copolymer comprises a polyvinylcaprolactam/vinylpyrrolidone/dimethylaminoethyl methacrylate copolymer, including it methylacrylate hydrolysis products, such as methacrylic acid and corresponding salts.

4. The cosmetic composition of claim 1 wherein said second non-crosslinking polyamide copolymer comprises a polyvinylcaprolactam/vinylpyrrolidone/dimethylaminoalkyl methacrymade copolymer; and further, wherein said second non-crosslinking polyamide copolymer comprises a polyvinylcaprolactam/vinylpyrrolidone/dimethylaminopropyl methacrylamide/quaternary methyacrylamidopropyl dimethylalkyl ammonium copolymer.

5. The cosmetic composition of claim 1 wherein said precipitated silica particles have a Median Particle Size from about 1 to about 20μ.

6.-7. (canceled)

8. The cosmetic composition of claim 1 wherein said precipitated silica particles have a Specific Surface Area (SSA) greater than about 300 M2/g.

9. The cosmetic composition of claim 1 wherein the weight ratio of said precipitated silica particles to said film-forming tensioning polymer system is from about 1:20 to about 1:2.

10.-12. (canceled)

13. The cosmetic composition of claim 1 wherein said precipitated silica particles comprise from about 0.1 to about 10 weight percent of said cosmetic composition.

14.-15. (canceled)

16. The cosmetic composition of claim 1 wherein said precipitated silica particles consist of precipitated hydrophilic silica particles.

17. The cosmetic composition of claim 1 wherein said precipitated silica particles comprise a mixture of precipitated hydrophilic silica particles and particles selected from the group consisting of hydrophobic particles, additional hydrophilic particles and combinations thereof.

18. The cosmetic composition of claim 17 wherein said hydrophobic particles comprise hydrophobically treated precipitated silica particles.

19. (canceled)

20. The cosmetic composition of claim 1 further comprising lipophilically or hydrophilically treated pigment powders.

21.-22. (canceled)

23. The cosmetic composition of claim 1 further comprising hydrophilically treated wax particles.

24.-26. (canceled)

27. The cosmetic composition of claim 1 wherein said cosmetic composition is in the form of a solution, an emulsion or a suspension.

28.-30. (canceled)

31. A water based system comprising the cosmetic composition of claim 1.

32. A skin foundation product comprising the cosmetic composition of claim 1.

33. A skin care product comprising the cosmetic composition of claim 1.

34. A hair styling product comprising the cosmetic composition of claim 1.

35. A mascara formulation comprising the cosmetic composition of claim 1.

36. A lip product comprising the cosmetic composition of claim 1.
